(19) United States
(12) Patent Application Publication
(10) Pub. No.: US 2024/0268989 A1
(43) Pub. Date: Aug. 15, 2024

(54) URINE COLLECTION CONTAINER BAFFLE STRUCTURES, AND RELATED SYSTEMS AND METHODS

(71) Applicant: PUREWICK CORPORATION, Covington, GA (US)

(72) Inventors: Adam Martin, Holly Springs, NC (US); Richard Abbott, Raleigh, NC (US); Nicholas Jardine, Holly Springs, NC (US)

(21) Appl. No.: 18/558,502

(22) PCT Filed: May 24, 2022
(86) PCT No.: PCT/US2022/030685

§ 371 Date: Nov. 1, 2023

Related U.S. Application Data
(60) Provisional application No. 63/193,235, filed on May 26, 2021.

(51) Int. Cl. A61F 5/451; A61F 5/44

(52) U.S. Cl. CPC A61F 5/451; A61F 5/4404

(57) ABSTRACT

Examples relate to urine collection container baffle structures, and related systems and methods. A urine collection system includes a urine collection device, a conduit, a canister, and a baffle structure. The urine collection device is configured to be positioned at least proximate to a urethra of a user. The conduit is in fluid communication with the urine collection device. The canister has an interior region, and the baffle structure is positioned or positionable in the interior region of the canister.

### CROSS-REFERENCE TO RELATED APPLICATIONS

This application claims priority to U.S. Provisional Patent Application No. 63/193,235 filed on May 26, 2021, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to urine collection container baffle structures, and related systems and methods. In an embodiment, a urine collection system includes a urine collection device, a conduit, a canister, and a baffle structure. The urine collection device is configured to be positioned at least proximate to a urethra of a user. The conduit is in fluid communication with the urine collection device. The canister includes an interior region, and the baffle structure is positioned or positionable in the interior region of the canister.

In an embodiment, a urine collection container includes a canister, a baffle structure, and a lid. The canister has an interior region and the baffle structure is positioned or positionable in the interior region of the canister. The lid is secured or securable to the canister and includes a collection port and a vacuum port.

In an embodiment, a method of collecting fluid includes positioning a baffle structure in an interior region of a canister. The method also includes positioning a fluid collection device at least proximate to a urethra of a user, the urine collection device being in fluid communication with the canister through a conduit. The method also includes collecting urine discharged by the user in the urine collection device. The method also includes drawing urine from the urine collection device through the conduit into the interior region of the canister, the urine contacting at least a portion of the baffle structure in the interior region of the canister.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a portable fluid collection system, according to an embodiment.

FIG. 1B is a front view of a urine collection system, according to an embodiment.

FIG. 2A is a side isometric view of a spiral baffle structure for a urine collection system, according to an embodiment.

FIG. 2B is a side isometric view of a urine collection container including a canister, a lid, and the spiral baffle structure of FIG. 2A positioned in a urine collection container, according to an embodiment.

FIG. 3A is a side isometric view of a horizontal transverse baffle structure for a urine collection system, according to an embodiment.

FIG. 3B is a side isometric view of a urine collection container including a canister, a lid, and the horizontal transverse baffle structure of FIG. 3A positioned in a urine collection container, according to an embodiment.

FIG. 4 is a side view of a baffle structure in a canister of a urine collection system, according to an embodiment.

FIG. 5 is a side view of a baffle structure in a canister of a urine collection system, according to an embodiment.

FIG. 6 is a flow diagram of a method for collecting fluid, according to an embodiment.

### DETAILED DESCRIPTION

Embodiments disclosed herein are related to urine collection containers and systems including baffle structures, and related methods. The devices and systems disclosed herein are configured to collect fluids from an individual. The fluids collected by the fluid collection devices may include at least one of urine, vaginal discharge, penile discharge, reproductive fluids, blood, sweat, or other bodily fluids.

In conventional urine collection systems, the sound of urine splashing during urine collection and/or canister movement is often embarrassing for users. For example, the urine may enter at the top of a canister and fall to the bottom of the canister or the urine level in the canister, increasing the splashing noise as the falling urine increases speed before contacting the bottom of the container or the urine level. The splashing noise may draw attention to the urine collection system during urine collection and/or movement by the user. Users of urine collection systems may have concerns about discretion when using urine collection systems, especially when the urine collection systems are used in ambulatory settings or other settings outside of a clinical setting. For example, some users may use a urine collection system while sitting in a wheelchair, with the urine collection canister positioned on a support on the wheelchair. As the wheelchair moves, the urine in the canister may create an embarrassing splashing noise, preventing the discretion desired by the user.

To improve conventional urine collection systems, baffle structures described herein are positioned or positionable in the canister of a urine collection systems. In at least one, some, or all embodiments of urine collection systems described herein, the baffle structure may be inserted into the canister to act as a baffling system that results in the technical effect of preventing the urine (or other fluids) from falling a longer distance and splashing in the canister, thereby preventing or inhibiting the distinct splashing noise common to conventional urine collection systems. The baffle structure may instead guide the urine through the baffle structure to the bottom of the canister and/or the urine level in the canister.

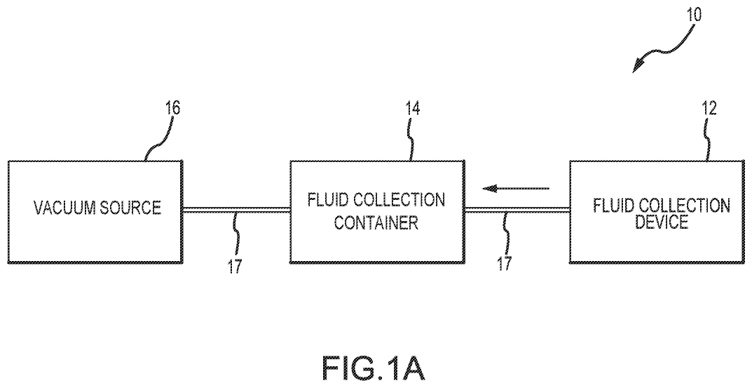

FIG. 1A is a block diagram of a fluid collection system 10, according to an embodiment. The fluid collection system 10 may be included in embodiments of fluid collection systems described herein. The system 10 includes a fluid (e.g., urine) collection device 12 (e.g., any of the fluid collection devices disclosed herein), a fluid (e.g., urine) collection container 14, and a vacuum source 16 (e.g., pump). The fluid collection device 12, the fluid collection container 14, and the vacuum source 16 may be fluidly coupled to each other via one or more conduits 17. For example, fluid collection device 12 may be operably coupled to one or more of the fluid collection container 14 or the vacuum source 16 via the conduit 17. In some embodiments, the vacuum source 16 may be secured directly to the fluid collection container 14. Fluid (e.g., urine or other bodily fluids) collected in the fluid collection device 12 may be removed from the fluid collection device 12 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the vacuum source 16 either directly or indirectly. The suction force may be applied indirectly via the fluid collection container 14. For example, the outlet of the conduit 17 may be disposed within or fluidly coupled to an interior region of the fluid collection container 14 and an additional conduit 17 may extend from the fluid collection container 14 to the vacuum source 16. Accordingly, the vacuum source 16 may apply suction to the fluid collection device 12 via the fluid collection container 14. The suction force may be applied directly via the vacuum source 16. For example, the outlet of the conduit 17 may be disposed within the vacuum source 16. An additional conduit 17 may extend from the vacuum source 16 to a point outside of the fluid collection device 12, such as to the fluid collection container 14. In such examples, the vacuum source 16 may be disposed between the fluid collection device 12 and the fluid collection container 14.

The fluid collection container 14 is sized and shaped to retain a fluid therein. The fluid collection container 14 may include a bag (e.g., drainage bag), a bottle, a canister, or a cup (e.g., collection jar), or any other enclosed container for storing bodily fluid(s) such as urine. In some examples, the conduit 17 may extend from the fluid collection device 12 and attach to the fluid collection container 14 at a first point therein. An additional conduit 17 may attach to the fluid collection container 14 at a second point thereon and may extend and attach to the vacuum source 16. Accordingly, a vacuum (e.g., suction) may be drawn through fluid collection device 12 via the fluid collection container 14. Fluid, such as urine, may be drained from the fluid collection device 12 using the vacuum source 16.

The vacuum source 16 may include one or more of a manual vacuum pump, and electric vacuum pump, a diaphragm pump, a centrifugal pump, a displacement pump, a magnetically driven pump, a peristaltic pump, or any source configured to produce a vacuum (e.g., negative pressure). The vacuum source 16 may provide a vacuum or suction to remove fluid from the fluid collection device 12. In some examples, the vacuum source 16 may be powered by one or more of a power cord (e.g., connected to a power socket), one or more batteries, or even manual power (e.g., a hand operated vacuum pump). In some examples, the vacuum source 16 may be sized and shaped to fit outside of, on, or within the fluid collection device 12. For example, the vacuum source 16 may include one or more miniaturized pumps or one or more micro pumps. The vacuum sources disclosed herein may include one or more of a switch, a button, a plug, a remote, or any other device suitable to activate the vacuum source 16.

FIG. 1B shows an example of a urine collection system 100, according to an embodiment. The urine collection system 100 may include a urine collection device 112, a first conduit 117a in fluid communication with an interior region of the urine collection device 112, a urine collection container, and a pump 116. The urine collection container may include a canister 114 and a lid 119 secured or securable to the canister 114. The canister 114 may include a top region 124 and a bottom region 134, and may at least partially define an interior region in fluid communication with the internal region of the urine collection device 112 via the first conduit 117a. For example, the lid 119 may include a urine port 139 to which the first conduit 117a may be detachably secured to provide fluid communication between urine collection device 112 and the interior region of the canister 114. A second conduit 117b may provide fluid communication between the interior region of the canister 114 and the pump 116. For example, the lid 119 may include a vacuum port 129 to which the second conduit 117b may be detachably secured to provide fluid communication between the pump 116 and the interior region of the canister. The vacuum port 129 and/or the urine port 139 may include an adapter detachably secured thereto to secure the respective conduit 117b, 117a to the vacuum port 129 and/or the urine port 139. In some embodiments, at least one (e.g., both) of the first conduit 117a and the second conduit 117b may be detachably connected or secured to the canister 114 effective to provide fluid communication with the interior region of the canister 114. For example, the canister may include at least one (e.g. both) of the urine port 139 and the vacuum port 129.

The urine collection device 112 may be configured to be positioned at least proximate to a urethra of a user. While the urine collection device 112 shown in FIG. 1B includes a female urine collection device, the urine collection device 112 may instead include a male urine collection device. PCT International Application No. PCT/US2019/029616, for example, describes various embodiments of both male and female fluid collection devices, the disclosure of which are incorporated in their entirety by this reference. Moreover, the urine collection device 112 may be interchangeable in the urine collection system 100 between different types, varieties, and sizes of male or female urine collection devices or other bodily fluid collection devices. Generally, the urine collection device 112 may include a surface sized to be positioned proximate or adjacent to the urethra and configured to wick urine or other fluids away from the user. Urine or other fluids may be wicked from the surface to a reservoir in the urine collection device 112.

The urine collection system 100 also includes the first conduit 117a in fluid communication with an interior region (e.g. reservoir) of the urine collection device 112 and the interior region of the canister 114. The first conduit 117a may be positioned or positionable between the urine collection device 112 and the canister 114. The urine collection system 100 also may include the second conduit 117b providing fluid communication between the pump 116 and the interior region of the canister 114. In some embodiments, the pump 116 may be secured directly to the canister 114, and the second conduit 117b may be absent from the urine collection system 100. The conduits 117a, 117b may include a flexible tube. In some embodiments, at least a portion of the first conduit 117a is substantially opaque, thereby inhibiting viewing of the urine within the first conduit 117a.

The canister 114 may be reusable and dishwasher safe, and may include a generally rigid material such as polycarbonate or glass. In some embodiments, the canister 114 may be disposable. The urine collection system 100 may be configured to accommodate different volumetric sizes of canisters 114. The canister 114 may be substantially cylindrical or frustoconical. In some embodiments, the canister may include other shapes and configurations, such as a generally square or rectangular side profile. In some embodiments, the lid 119 having multiple ports 129, 139 and/or adapters for attachment of the conduits 117a, 117b thereto may be secured or securable to the canister 114. In some embodiments, the lid 119 may include additional ports or adapters than those shown in FIG. 1B. Except for the ports and/or adapters for attachment of the conduits 117a, 117b, the canister 114 may be sealed and airtight such that the urine collected in the canister 114 does not leak or spill.

The pump 116 is in fluid communication with the interior region of the canister 114 and is configured to pull a vacuum on the interior region of the canister 114 effective to draw the urine from the urine collection device 112 through the first conduit 117a into the canister 114. The pump 116 may be secured directly to the canister 114, or the conduit 117b may fluidly couple the pump 116 with the interior region of the canister 114. The pump 116 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 116 may provide a vacuum or suction to remove fluid from the fluid collection device 112. In some examples, the pump 116 may be powered by one or more batteries or other power sources. The pump 116 may be included in a housing that also supports the canister 114. In some examples, the pump 116 may be sized and shaped to fit within a container support on a wheelchair. In some embodiments, the pump 116 may include a wall-mounted pump. The pump 116 may include one or more of a switch, a button, a plug, a remote, or any other device suitable to activate the pump 116.

The urine collection system 100 may include a baffle structure positioned or positionable in the interior region of the canister 114. In at least one, some or all embodiments of baffle structures described herein, the configuration of the baffle structure results in the technical effect of improving discretion during urine collection and/or filling of the canister. For example, in contrast to conventional urine collection systems, at least one, some, or all embodiments of baffle structures described herein prevent urine or other fluid from splashing into the canister 114 as the urine is drawn from the user and dispensed into the canister 114. In conventional systems, urine splashing into the canister draws attention to the urine collection system due to the noise generated by the urine splashing in the container during and after filling of the container. In urine collection systems designed for ambulatory patients, noise from urine splashing during collection and movement is increased and/or causes additional embarrassment for users.

To improve the urine collection system 100, a baffle structure may be positioned or positionable in the interior region of the canister. The baffle structure may reduce the distance urine freefalls as the urine enters the canister 114, thereby inhibiting amplification of the splashing noise. To reduce the distance the urine freefalls in the canister 114, the baffle structure may guide the urine the bottom region 134 of the canister 114 (or the current urine level in the canister 114) to prevent or minimize the splashing noise in the canister. Many embodiments of baffle structures described herein may be removable from the canister 114 and reusable in a different canister 114. In some embodiments, the baffle structures may be integrally formed (e.g., molded) with the canister 114.

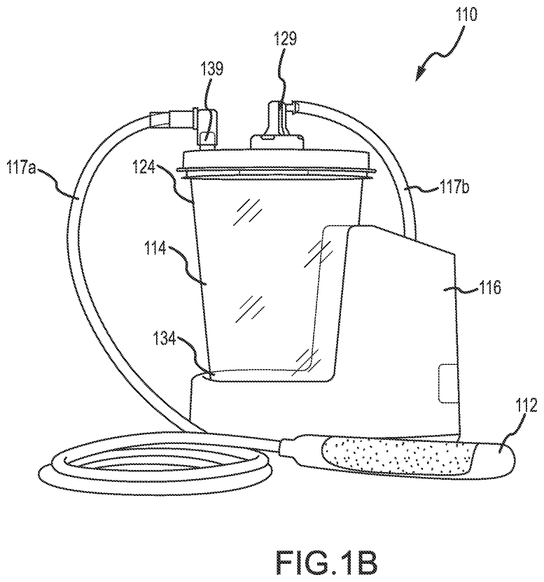

FIG. 2A is an isometric side view of a spiral baffle structure 200 and FIG. 2B is an isometric view of a urine collection container 250 including the lid 119 secured to the top region 124 of the canister 114 and the baffle structure 200 positioned in the interior region of the canister 114, according to an embodiment. The baffle structure 200 may include one or more baffles. For example, the one or more baffles of the baffle structure 200 may include at least one plate 210 having or defining a plurality of holes 212 extending therethrough and a helical body 220 secured to the plate 210. When the baffle structure 200 is positioned in the canister 114, the helical body 220 extends from the bottom region 134 at least partially towards the top region 124 of the canister 114 and the plate 210 is positioned proximate the top region 124 of the canister 114. In some embodiments, a distal end 222 of the helical body 220 opposite to the plate 210 may rest on the bottom of the canister 114.

In some embodiments, the canister 114 includes one or more sidewalls 125 having an inner perimeter 123 at least partially defining the interior region of the canister 114. The plate 210 may include an outer periphery 215 generally complementary to the inner perimeter 123 of the one or more sidewalls 125 at the top region 124 of the canister 114. The plate 210 may be sized such that the outer periphery 215 interfaces or is adjacent to the inner perimeter 123, or is spaced from the inner perimeter 123 of the canister 114. In some embodiments, the helical body 220 includes an outer periphery 225 generally complementary to the inner perimeter 123 of the one or more sidewalls 125 at least partially between the top region 124 and the bottom region 134 of the canister 114. The helical body 220 may be sized such that the outer periphery 225 interfaces or is adjacent to the inner perimeter 123, or is spaced from the inner perimeter 123 of the canister 114. For example, the canister 114 may include a frustoconical shape that narrows between the top region 124 and the bottom region 134. The plate 210 may be substantially circular and the helical body 220 may include a width that decreases as the helical body 220 increases in distance from the plate 210, generally complementary to the narrowing of the canister 114 (e.g., the width or diameter of the helical body 220 at the distal end 222 is less than the width or diameter of the helical body 220 at the top end proximate to the plate 210). In some embodiments, the plate 210 may include a shape other than a circle, such as an oval, a triangle, a rectangle, a pentagon, a hexagon, etc. A top or bottom profile of the helical body 220 also may include a shape other than a circle, such as an oval, a triangle, a rectangle, a pentagon, a hexagon, etc. The shape of the plate 210 and/or the helical body 220 may be generally complementary to the shape of the container 114.

In some embodiments, the plate 210 may include four theoretical quadrants 211 or regions of substantially equal dimensions, and the plurality of holes 212 may be positioned on the plate 210 such that at least one hole 212 of the plurality of holes 212 is positioned within each quadrant 211 of the plate 210. In some embodiments, the plurality of holes 212 may be positioned on the plate 210 such that multiple holes 212 of the plurality of holes 212 are positioned within each quadrant 211 of the plate 210.

The plate 210 also may define an opening 232 positioned on the plate 210 to generally align with and/or be positioned directly below the vacuum port 129 on the lid 119 when the lid 119 is secured to the canister 114. For example, the vacuum port 129 may be positioned generally centrally on the lid 119 and the opening 232 may be positioned generally centrally on the plate 210. In some embodiments, the vacuum port 129 may be positioned off-center on the lid 119 and the opening 232 may be positioned to align with or be directly below the vacuum port 129 when the vacuum port 129 is off-center. The opening 232 may allow the necessary air in the canister 114 to be available for suction by the pump 116 through the vacuum port 129 when the pump 116 is activated. In some embodiments, the opening 232 has an area or diameter greater than an area or diameter of each hole 212 of the plurality of holes 212. The plate 210 also may include a lip 230 at least partially defining the opening 232 and extending from the plate 210 distal to the helical body 220.

The plate 210 also may be spaced from the lid 119 when the baffle structure 200 is positioned in the interior region of the canister 114. For example the plate 210 may be spaced about 0.64 cm to about 5.1 cm from lid 119, about 1.3 cm to about 2.5 cm from the lid 119, about 2.5 cm to about 3.8 cm from the lid 119, about 3.8 cm to about 5.1 cm from the lid 119, about 0.64 cm to about 1.3 cm from lid 119, about 1.3 cm to about 1.9 cm from lid 119, about 1.9 cm to about 2.5 cm from the lid 119, about 2.5 cm to about 3.2 cm from the lid 119, about 3.2 cm to about 3.8 cm from the lid 119, about 3.8 cm to about 4.4 cm from the lid 119, about 4.4 cm to about 5.1 cm from the lid 119, less than about 5.1 cm from the lid 119, less than about 3.8 cm from the lid 119, less than about 2.5 cm from the lid 119, less than about 1.9 cm from the lid 119, or less than about 1.3 cm from the lid 119.

In use, when urine or other fluid enters the interior region of the canister 114 through the urine port 139 on the lid 119, the urine only falls a short distance (e.g., distance that is less than a distance to the bottom of the canister and/or distance that is less than a distance until the urine level in the canister 114) until the urine contacts the plate 210 of the baffle structure 200. The urine may then fall through one or more holes of the plurality of holes 212 and fall a short distance until the urine contacts the helical body 220. The urine may then continue to flow down the helical body 220 until the urine reaches the bottom of the canister 114 or the urine level in the canister. At least one, some, or all embodiments of the baffle structure 200, then, results in the technical effect of reducing the distance of freefall of urine in entering the canister 114 and/or moving through the canister, thus reducing or preventing a splashing noise in the canister 114 as the urine is collected. The baffle structure 200 also may prevent sloshing and splashing of urine held in the canister 114 as the canister 114 is moved.

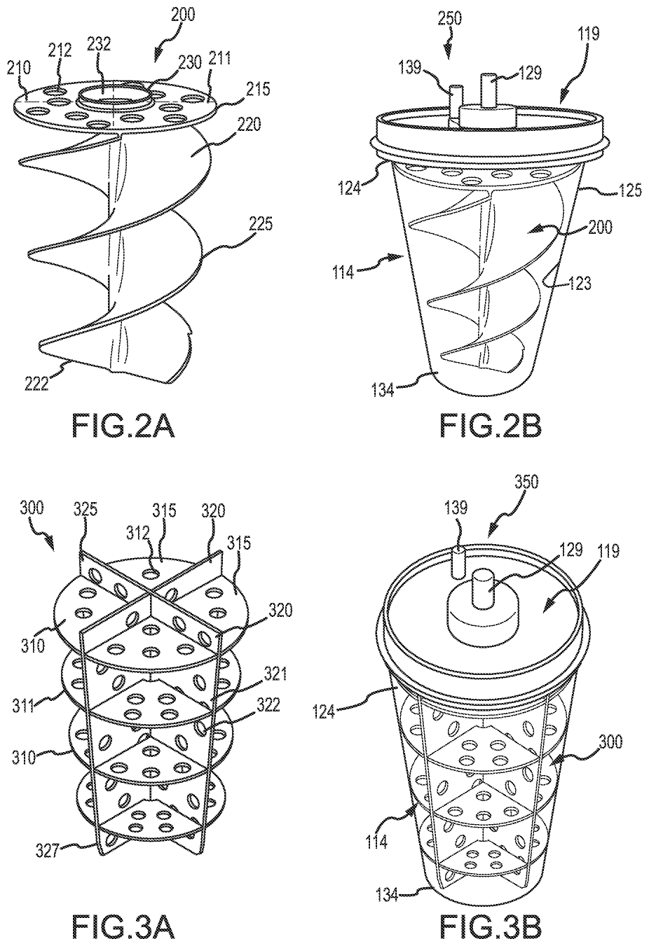

FIG. 3A is an isometric side view of a horizontal transverse baffle structure 300 and FIG. 3B is an isometric view of a urine collection container 350 including the baffle structure 300 positioned in the canister 114 and the lid 119 secured to the top region 124 of the canister 114, according to an embodiment. The baffle structure 300 may include one or more baffles. For example, the one or more baffles of the baffle structure 300 may include one or more (e.g., two, three, four, five, six, etc.) walls 320 and a plurality of a plates 310 secured and spaced along the one or more walls 320. Each plate 310 of the plurality of plates 310 may include a plurality of holes 312. In some embodiments, the plurality of plates 310 includes at least a top plate, a bottom plate, and one or more (e.g., two, three, four, etc.) intermediate plates positioned between the top plate and the bottom plate.

The one or more walls 320 may be positioned and/or secured to the plurality of plates 310 to divide each plate of the plurality of plates 310 into multiple plate regions 315. For example, the baffle structure 300 includes four walls 320 that divide each plate 310 into four different plate regions 315. At least one hole 312 of the plurality of holes 312 may be positioned in each plate region 312 of the multiple plate regions 315. In some embodiments, multiple holes 312 may be positioned in each plate region 315 of the multiple plate regions 315. The multiple holes 312 in each plate region 315 may be positioned such that alignment of the multiple holes 312 on adjacent plates 310 is absent (e.g., the multiple holes 312 in each plate region 315 are not aligned with the multiple holes 312 in each plate region 315 of an adjacent plate 310, or the multiples holes 312 in each plate region 314 are misaligned with the multiple holes 312 in each plate region 315 of an adjacent plate 310). For example, the multiple holes 312 on the top plate 310 may be arranged in a generally triangular or diamond formation, and the multiple holes 312 on the intermediate plate 310 adjacent to the top plate 310 may be arranged in a generally square formation. In any event, each hole of the plurality of holes 312 may not be aligned with any holes of the plurality of holes 312 on an adjacent plate.

In some embodiments, one or more (e.g., some or all) walls 320 of the multiple of walls 320 may include a plurality of holes 322. For example, the plurality of plates 310 may divide each wall 320 of the multiple walls 320 into multiple wall regions 321. One or more holes 322 of the plurality of holes 322 may be positioned in each wall region 321 of the multiple wall regions 321. In some embodiments, one or more (e.g., all) walls 320 of the multiple walls 320 includes a top portion 325 extending above the top plate of the plurality of plates 310 and a bottom portion 327 extending below the bottom plate of the plurality of plates 310. At least one (e.g., both) of the top portion 325 and the bottom portion 327 may include at least one hole 322 of the plurality of holes.

Turning to FIG. 3B, in some embodiments, the baffle structure 300 is removably positionable in the interior region of the canister 114. When positioned in the interior region of the canister 114, the top plate of the plurality of plates 310 is positioned proximate to the top region 124 and the bottom plate of the plurality of plates 310 is positioned proximate to the bottom region 134 of the canister 114. The plurality of plates 310 may include an outer periphery 311 generally complementary to the inner perimeter 123 of the one or more sidewalls 125 of the canister 114 when the baffle structure 300 is positioned in the interior region of the canister 114. In some embodiments, the canister 114 includes a frustoconical shape that narrows between the top region 124 and the bottom region 134. Each plate of the plurality of plates 310 may be substantially circular, with the top plate having a diameter greater than a diameter of the bottom plate. The urine collection container 350 may include the lid 119 removably secured or securable to the canister 114 with the baffle structure 300 positioned in the interior region of the canister 114. In some embodiments, the plurality of plates 310 may include a shape other than a circle, such as an oval, a triangle, a rectangle, a pentagon, a hexagon, etc. The shape of the plurality of plates 310 may be generally complementary to the shape of the container 114.

In use, urine entering the canister may fall only a short distance before contacting the top plate of the plurality of plates 310. The urine may then pass through one or more holes of the plurality of holes 312 on the top plate and fall another short distance before contacting the adjacent intermediate plate of the plurality of plates 310. The urine may then pass through one or more holes of the plurality of holes 312 on the adjacent intermediate plate of the plurality of plates 310 and fall another short distance before contacting an adjacent plate of the plurality of plates 310, and so one. In at least one, some, or all embodiments, the plurality of plates 310, then, result in the technical effect of reducing the distance of freefall of urine in entering the canister 114 and/or moving through the canister 114, thus reducing or preventing a splashing noise in the canister 114 as the urine is collected. The walls 320 also prevent or inhibit sloshing and splashing noises of urine in the canister 114 as the canister is moved.

Urine collection systems including no baffle structure, the baffle structure 200, and the baffle structure 200 were tested comparatively to determine the effect of the baffle structures 200, 300 on the noise generated during urine collection and movement of the canister 114. It was observed that the baffle structures 200, 300 decreased splashing noises during filling of the canister 114 by reducing distance of fluid freefall into pooled liquid collected in the canister 114.

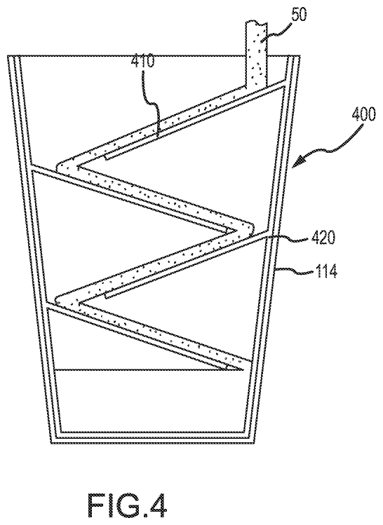

Turning to FIG. 4, the canister 114 is shown with a baffle structure 400 positioned in the interior region of the canister 114, according to an embodiment. Although the canister 114 is shown without the lid 119, the lid 119 may be secured to the canister with the baffle structure 400 positioned therein effective to form a urine collection container including the baffle structure 400. In some embodiments, the baffle structure 400 includes a plurality downward sloping walls 410 that partially overlap with an adjacent wall of the plurality of downward sloping walls 410. As urine 50 enters the interior region of the canister 114, the urine 50 may first contact a top downward sloping wall of the plurality of downward sloping walls. The urine 50 may then flow to the end of the top downward sloping wall and fall to an adjacent downward sloping wall of the plurality of downward sloping walls 410. The urine may then flow down to the end of the adjacent downward sloping wall and fall onto an additional adjacent downward sloping wall of the plurality of downward sloping walls 410 below the adjacent downward sloping wall. This process may continue through the plurality of downward sloping wall 410 until the urine 50 reaches the bottom of the canister 114 and/or the level of urine 50 held in the canister 114. The plurality of downward sloping walls 410 may be positioned such there is no empty space in the canister 114 extending continuously from the top of the interior region to the bottom of the interior region.

In some embodiments, the baffle structure 400 may be removably positionable or positioned in the interior region of the canister and may include one or more a supports 420. The plurality of downward sloping walls 410 may extending inward and downward from the one or more supports 420 when the baffle structure 400 is positioned in the interior region of the canister 114.

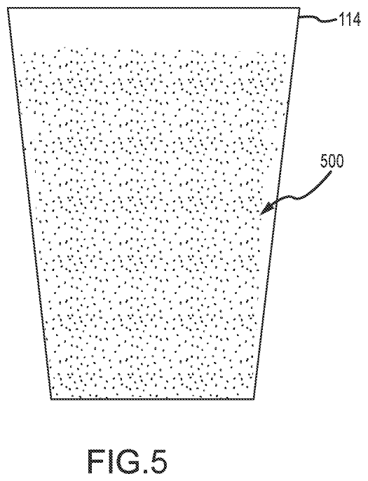

Turning to FIG. 5, in some embodiments a baffle structure may include a plurality of beads 500 positioned or positionable in the interior region of the canister 114. The plurality of beads 500 may be one or more of silica beads, glass beads, carbon beads, carbon beads, clay beads, other desiccant beads, other structures such as shavings or helical pieces of material, or combinations thereof. The plurality of beads 500 may be positioned in the interior region of the canister 114 to a predetermined or desired level.

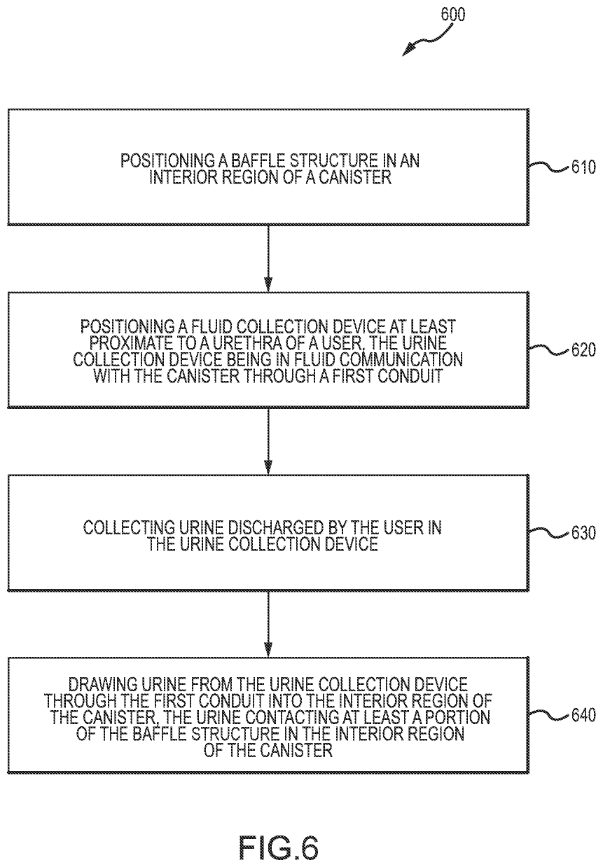

FIG. 6 is a flow diagram of a method 600 for collecting fluid, according to an embodiment. The method 600 includes an act 610 of positioning a baffle structure having one or more baffles in an interior region of a canister. In some embodiments, the method 600 includes an act 620 of positioning a fluid collection device at least proximate to a urethra of a user, the urine collection device being in fluid communication with the canister through a first conduit. In some embodiments, the method 600 includes an act 630 of collecting urine discharged by the user in the urine collection device. In some embodiments, the method 600 includes an act 640 of drawing urine from the urine collection device through the first conduit into the interior region of the canister, the urine contacting at least a portion of the baffle structure in the interior region of the canister.

The baffle structure in the method 600 may include any baffle structure described herein. In some embodiments, the one or baffles of the baffle structure of the method 600 may include a plate having a plurality of holes and a helical body secured to the plate and the act 610 includes positioning the plate proximate to a top region of the canister with the helical body extending at least partially between the top region and a bottom region of the canister. In some embodiments, the one or more baffles of the baffle structure of the method 600 may include one or more walls and a plurality of a plates secured and spaced along the one or more walls, each plate of the plurality of plates including a plurality of holes. The act 610 may include positioning a top plate of the plurality of plates proximate to a top region of the canister and a bottom plate of the plurality of plates proximate to a bottom region of the canister.

The acts of the method 600 described above are for illustrative purposes. For example, the acts of the method 600 can be performed in different orders, split into multiple acts, modified, supplemented, or combined. In an embodiment, one or more of the act of the method 600 can be omitted from the method 600. Any of the acts of the method 600 can include using any of the baffle structures disclosed herein.

As used herein, the term “about” or “substantially” refers to an allowable variance of the term modified by “about” or “substantially” by +10% or +5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A urine collection system, comprising:
a urine collection device configured to be positioned at least proximate to a urethra of a user;
a first conduit in fluid communication with the urine collection device;
a canister having a top region, a bottom region, and one or more sidewalls extending at least partially between the top region and the bottom region and including an inner perimeter that defines an interior region of the canister in fluid communication with the first conduit; and
a baffle structure positioned or positionable in the interior region of the canister, the baffle structure including one or more plates having a plurality of holes extending therethrough and an outer periphery shaped generally complementary to the inner perimeter of the one or more sidewalls of the canister, wherein the one or more plates include at least a top plate positioned at least proximate to the top region of the canister when the baffle structure is positioned in the interior region of the canister.

2. The urine collection system of claim 1, wherein:
the baffle structure is removably positioned in the interior region of the canister and includes the top plate having the plurality of holes and a helical body secured to the top plate, the helical body extending at least partially between the top region and the bottom region and including an outer periphery generally complementary to the inner perimeter of the one or more sidewalls at least partially between the top region and the bottom region;
the canister includes a frustoconical shape that narrows between the top region and the bottom region;
the top plate is substantially circular; and
the helical body includes a width that decreases as the helical body increases in distance from the top plate.

3. (canceled)

4. (canceled)

5. (canceled)

6. The urine collection system of claim 2, wherein the plurality of holes in the top plate are positioned on the top plate such that at least one hole of the plurality of holes is positioned within each quadrant of the top plate.

7. The urine collection system of claim 2, wherein the top plate defines an opening having an area or diameter greater than an area or diameter of each hole of the plurality of holes.

8. The urine collection system of claim 7, wherein the top plate includes a lip extending distal to the helical body and defining the opening.

9. The urine collection system of claim 7, further comprising a lid removably secured or securable to the canister and having a collection port in fluid communication with the first conduit and a vacuum port configured to provide fluid communication between a vacuum source and the interior region of the canister via a second conduit.

10. The urine collection system of claim 9, wherein the top plate is spaced from the lid and the vacuum port and the opening in the top plate are substantially aligned with the vacuum port positioned over the opening in the top plate when the baffle structure is positioned in the interior region and the lid is secured to the canister.

11. The urine collection system of claim 10, wherein the vacuum port is positioned generally centrally on the lid and the opening is positioned generally centrally on the top plate.

12. The urine collection system of claim 1, wherein the baffle structure is removably positioned in the interior region of the canister and includes one or more walls and a plurality of plates secured and spaced along the one or more walls, the plurality of plates including the top plate and one or more additional plates, each plate of the plurality of plates including the plurality of holes.

13. The urine collection system of claim 12, wherein:
the plurality of plates includes at least the top plate, a bottom plate, and one or more intermediate plates positioned between the top plate and the bottom plate; and
the one or more walls include multiple walls positioned to divide each plate of the plurality of plates into multiple plate regions.

14. The urine collection system of claim 13, wherein each plate of the plurality of plates includes at least one hole of the plurality of holes positioned in each plate region of the multiple plate regions.

15. The urine collection system of claim 14, wherein each plate of the plurality of plates includes multiple holes of the plurality of holes positioned in each plate region of the multiple plate regions, the multiples holes being positioned such that alignment of the multiple holes on adjacent plates of the plurality of plates is absent.

16. The urine collection system of claim 13, wherein each wall of the multiple walls includes a plurality of holes.

17. The urine collection system of claim 16, wherein:
the plurality of plates divide each wall of the multiple walls into multiple wall regions; and
one or more holes of the plurality of holes on each wall of the multiple walls are positioned in each wall region of the multiple wall regions.

18. The urine collection system of claim 13, wherein each wall of the multiple walls includes a top portion extending above the top plate and a bottom portion extending below the bottom plate.

19. The urine collection system of claim 18, wherein the top portion and the bottom portion of each wall of the multiple walls each include at least one hole.

20. (canceled)

21. (canceled)

22. The urine collection system of claim 13, wherein:
the canister includes a frustoconical shape that narrows between the top region and the bottom region;
each plate of the plurality of plates is substantially circular; and
the top plate includes a diameter greater than a diameter of the bottom plate.

23. The urine collection system of claim 1, further comprising a lid removably secured or securable to the canister and having a collection port in fluid communication with the first conduit and a vacuum port configured to provide fluid communication between a vacuum source and the interior region of the canister via a second conduit.

24. (canceled)

25. (canceled)

26. (canceled)

27. The urine collection system of claim 1, further comprising a pump in fluid communication with the canister and configured to pull an at least partial vacuum on the interior region of the canister effective to draw urine from the urine collection device through the first conduit into the canister.

28. A urine collection container, comprising:
a canister having a top region, a bottom region, and one or more sidewalls extending at least partially between the top region and the bottom region and including an inner perimeter that defines an interior region of the canister;
a baffle structure positioned or positionable in the interior region of the canister, the baffle structure including one or more plates having a plurality of holes extending therethrough and an outer periphery shaped generally complementary to the inner perimeter of the one or more sidewalls of the canister, wherein the one or more plates include at least a top plate positioned at least proximate to the top region of the canister when the baffle structure is positioned in the interior region of the canister; and
a lid secured or securable to the canister, the lid including a collection port and a vacuum port.

29. The urine collection container of claim 28, wherein:
the baffle structure is removably positioned in the interior region of the canister and includes the top plate having the plurality of holes and a helical body secured to the top plate, the helical body extending at least partially between the top region and the bottom region and including an outer periphery generally complementary to the inner perimeter of the one or more sidewalls at least partially between the top region and the bottom region;
the canister includes a frustoconical shape that narrows between the top region and the bottom region;
the top plate is substantially circular; and
the helical body includes a width that decreases as the helical body increases in distance from the top plate.

30. (canceled)

31. (canceled)

32. (canceled)

33. The urine collection container of claim 29, wherein the plurality of holes in the top plate are positioned on the top plate such that at least one hole of the plurality of holes is positioned within each quadrant of the plate.

34. The urine collection container of claim 29, wherein the top plate defines an opening having an area or diameter greater than an area or diameter of each hole of the plurality of holes.

35. The urine collection container of claim 34, wherein the top plate includes a lip extending distal to the helical body and defining the opening.

36. The urine collection container of claim 35, wherein the top plate is spaced from the lid and the vacuum port and the opening in the top plate are substantially aligned with the vacuum port positioned over the opening in the top plate when the baffle structure is positioned in the interior region and the lid is secured to the canister.

37. The urine collection container of claim 36, wherein the vacuum port is positioned generally centrally on the lid and the opening is positioned generally centrally on the top plate.

38. The urine collection container of claim 28, wherein the baffle structure is removably positioned in the interior region of the canister and includes one or more walls and a plurality of plates secured and spaced along the one or more walls, the plurality of plates including the top plate and one or more additional plates, each plate of the plurality of plates including the plurality of holes.

39. The urine collection container of claim 38, wherein:
the plurality of plates includes at least the top plate, a bottom plate, and one or more intermediate plates positioned between the top plate and the bottom plate; and
the one or more walls include multiple walls positioned to divide each plate of the plurality of plates into multiple plate regions.

40. The urine collection container of claim 39, wherein each plate of the plurality of plates includes at least one hole of the plurality of holes positioned in each plate region of the multiple plate regions.

41. The urine collection container of claim 40, wherein each plate of the plurality of plates includes multiple holes of the plurality of holes positioned in each plate region of the multiple plate regions, the multiples holes being positioned such that alignment of the multiple holes on adjacent plates of the plurality of plates is absent.

42. The urine collection container of claim 39, wherein each wall of the multiple walls includes a plurality of holes.

43. The urine collection container of claim 42, wherein:
the plurality of plates divide each wall of the multiple walls into multiple wall regions; and
one or more holes of the plurality of holes on each wall of the multiple walls are positioned in each wall region of the multiple wall regions.

44. The urine collection container of claim 39, wherein each wall of the multiple walls includes a top portion extending above the top plate and a bottom portion extending below the bottom plate.

45. The urine collection container of claim 44, wherein the top portion and the bottom portion of each wall of the multiple walls each include at least one hole.

46. (canceled)

47. (canceled)

48. The urine collection container of claim 39, wherein:
the canister includes a frustoconical shape that narrows between the top region and the bottom region;
each plate of the plurality of plates is substantially circular; and
the top plate includes a diameter greater than a diameter of the bottom plate.

49. (canceled)

50. (canceled)

51. (canceled)

52. A method of collecting fluid, the method comprising:
positioning a baffle structure in an interior region of a canister, the baffle structure including one or more plates having a plurality of holes extending therethrough and an outer periphery shaped generally complementary to an inner perimeter of one or more sidewalls of the canister, wherein the one or more plates include at least a top plate positioned at least proximate to the top region of the canister;
positioning a fluid collection device at least proximate to a urethra of a user, the urine collection device being in fluid communication with the canister through a first conduit;
collecting urine discharged by the user in the urine collection device; and
drawing urine from the urine collection device through the first conduit into the interior region of the canister, the urine contacting at least a portion of the one or more baffles in the interior region of the canister.

53. The method of claim 52, wherein the baffle structure includes the plate having the plurality of holes and a helical body secured to the plate and positioning a baffle structure in an interior region of a canister includes positioning the plate proximate to a top region of the canister with the helical body extending at least partially between the top region and a bottom region of the canister.

54. The method of claim 52, wherein the baffle structure includes one or more walls and a plurality of plates secured and spaced along the one or more walls, the plurality of plates including the top plate and one or more additional plates, each plate of the plurality of plates including the plurality of holes, and wherein positioning a baffle structure in an interior region of a canister includes positioning the top plate of the plurality of plates proximate to a top region of the canister and a bottom plate of the plurality of plates proximate to a bottom region of the canister.
